(19) United States
(12) Patent Application Publication
(10) Pub. No.: US 2024/0149018 A1
(43) Pub. Date: May 9, 2024

(54) TUBE DEVICE AND METHOD FOR SELECTIVELY STIFFENING A TUBE DEVICE

(71) Applicant: SoftRail Medical AG, Basel (CH)

(72) Inventors: Alexander VON WEYMARN, Frauenfeld (CH); Thomas GERIG, Burgdorf (CH)

(73) Assignee: SoftRail Medical AG, Basel (CH)

(21) Appl. No.: 18/549,723

(22) PCT Filed: Feb. 28, 2022
(86) PCT No.: PCT/EP2022/054918

§ 371 Date: Sep. 8, 2023

(30) Foreign Application Priority Data
Mar. 9, 2021 (EP) 21161563.8

(51) Int. Cl. A61M 25/00; A61L 29/02; A61L 29/04; A61L 29/08; A61M 25/09; A61M 29/00

(52) U.S. Cl. CPC A61M 25/0045; A61L 29/02; A61L 29/04; A61L 29/085; A61M 25/005; A61M 25/0071; A61M 25/0097; A61M 25/09; A61M 29/00; A61M 2025/0065; A61M 2205/0216

(57) ABSTRACT

The invention relates to a tube device (100), in particular a lock-type device for insertion in a body lumen, that can be reversibly stiffened under the effect of pressure by a concentric arrangement of an inner (1) and outer tube (4), and a stiffening layer (3), the stiffening layer exerting pressure on the outer tube and thus producing high friction.

The invention is directed to a tubing apparatus and a method for selectively stiffening a tubing apparatus.

Minimally invasive surgical procedures, such as the treatment of aneurysms with the placement of stents, dilate, lyse and embolize, use tubing apparatuses that can be inserted into the body through the vessels.

In many cases, the catheters, which are often inserted through the inguinal artery, must be able to travel long distances in the patient's body and make turns along the blood vessels.

Furthermore, the treatment must allow for an internal space within the catheter for insertion of tools and/or a guide wire and/or surgical treatment elements such as stents, coils or the like.

In these medical treatments, it is particularly important to be able to work as precisely and minimally invasively as possible, as the health of the person being treated can be affected.

Since the tubing apparatus, e.g. a catheter, must be elastically deformable in order to adapt to the coils of the vessel, to be steered and to be inserted, it can very easily slip or be deflected. On the one hand, this is very dangerous, as it makes it difficult to work precisely and the treatment can be drawn out. On the other hand, the flexible catheter increases the risk of patient harm or even death. For example, an aneurysm, or calcium deposits present in the vessels, can become detached due to unfavorable slippage and cause serious damage. In many cases it is necessary to change to a different catheter, so that the material consumption is very high and so are the costs.

It is therefore an object of the present invention to overcome these disadvantages of the prior art and to present a device with which minimally invasive surgical procedures can be performed with as little risk as possible, can be performed quickly, and costs can be kept low.

The task is solved by a tubing apparatus according to the independent claims of the invention.

A tubing apparatus, in particular a gate apparatus, for insertion into a body passage, comprises a stiffening section with an inner tube, as well as a stiffening layer and an outer tube. The stiffening layer is arranged concentrically outside the inner tube, and the outer tube is arranged concentrically outside the stiffening layer. The inner tube and the outer tube are radially rigid and the stiffening layer is radially movable. Pressure can be built up between the inner tube and the stiffening layer in such a way that the stiffening layer can be pressed from the inside against the outer tube and thus stiffen the stiffening section under pressure.

The tubing apparatus thus has the advantage that it can be reversibly stiffened by a pressure during use. The preferred pressure is essentially in a range of 3-40 bar, in particular essentially at 16 bar, but other pressure ranges are also conceivable as long as stiffening is ensured.

Radially rigid in the sense of the invention means that the radially rigid layer can be expanded only minimally by pressure in the radial direction, so that pressure can be applied at all. Radially movable means that the layer is not radially rigid. Radially rigid does not exclude that the layer is flexible in other directions.

The pressure can be checked and released preferably by an external device, which may include further protective measures, such as shutdown in case of pressure drop due to a leak, applied, or manually applied.

At this point it should be noted that the pressure difference to the human body must be taken into account. For this reason, the tubing apparatus is designed so that a high pressure can be supplied and discharged. Preferably, a pressure above atmospheric pressure is used and not a vacuum or negative pressure. This has the advantage that the pressure difference is much higher, since the negative pressure is limited to a maximum value of 1 bar.

The formation of a stiffener facilitates the fixation of the tubing apparatus in the desired location and allows for a more precise procedure that increases patient safety, is more feasible to perform, and ensures faster work.

In the stiffened state, the pressure can also be reduced again and the stiffening can be reversed.

The stiffenable area of the tubing apparatus can also vary in length, since the stiffening layer does not have to be formed over the entire length of the tubing apparatus. Accordingly, certain portions of the tubing apparatus may be formed to be stiffenable. The stiffening section of the tubing apparatus may comprise the entire tubing apparatus or only a partial section. Provided that only a partial section of the tubing apparatus is formed by the stiffening section, the remainder of the tubing apparatus is not stiffenable.

In the innermost part of the tubing apparatus is an inner space surrounded by the inner tube, so that a lumen is created in the inner space, which in proper use is not reduced by kinks or pressure on the tubing apparatus. This lumen has an opening to each end of the tubing apparatus.

For this purpose, it is important that the inner and outer tubes are radially rigid. This radial rigidity ensures that there is no or only minor expansion and contraction of the inner and outer tubes. This ensures that no kinking of the tubing can occur, which would severely constrict the interior and limit the insertion and use of apparatus in the tubing apparatus.

Nevertheless, high flexibility and bendability of the tubing apparatus is possible so that the tubing apparatus can complete turns along the vessels and branches of the vessels.

The radial mobility of the stiffening layer, on the other hand, is important so that the stiffening section can be stiffened by pressure, since this is only possible by form fit and/or force fit if the stiffening layer can be pressed against the outer tube from the inside by the built-up pressure.

In the tubing apparatus, an expandable, in particular elastic and/or unfoldable, pressure tube can preferably be arranged between the inner tube and the stiffening layer.

The pressure tube can be subjected to pressure, thus pressing the stiffening layer against the outer tube and stiffening the stiffening section of the tubing apparatus. This arrangement has the advantage that the pressure tube also provides a safety mechanism.

It should be noted that this arrangement is advantageous for potential leaks in the hose. Patient safety would be ensured despite a leak, as the pressure is built up between the inner tube and the stiffening layer in the pressure tube. Thus, a double leak would have to occur for the pressure to escape via the outer tube.

Furthermore, pressure can thus be applied to the stiffening layer without the need for the stiffening layer to have a dense layer. It is also conceivable to have a stiffening layer consisting of spirally arranged elements or a stiffening layer forming a closure with a complementarily formed inner surface of the outer tube for stiffening the tubing apparatus. Preferably, the stiffening layer is nonetheless not monolithically manufactured. This preferred design of the tubing apparatus allows the stiffening layer to be more flexible in that it can have a non-enclosed structure, construction or surface, such as perforated, grooved, spiral, mesh, interwoven or netted. This can have an advantageous effect on the stiffening of the stiffening section, as the stiffening section can thus be stiffened more easily during severe bending. The pressure tube can expand when subjected to pressure, and preferably comprises elastically deformable material, and/or a foldable material, such as a plastic.

Nylon, polyethylene terephthalate and/or polyether block amide are particularly suitable for an expandable and retractable pressure tube.

In particular, the pressure tube advantageously comprises stretchable elastomers or other thermoplastic plastic material, such as silicone and/or thermoplastic elastomers and/or foldable plastics. These materials have a high biocompatibility, are non-toxic, are inexpensive, and are preferably very elastically deformable.

Particularly advantageous is a pressure tube that can have a collapsed state without the action of additional pressure and can have an unfolded state when pressure is applied. Such an unfoldable pressure tube ensures a fixed maximum and minimum size when proper pressures are applied, and stiffening with maximized tightness can be ensured.

The stiffening layer may comprise at least two stabilizing elements, wherein the orientation of the stabilizing elements has at least a partial vector in the longitudinal direction of the tubing apparatus and the stabilizing elements are preferably displaceable relative to each other.

The individual stabilizing elements can be movable, in particular displaceable, relative to one another in the unstiffened state. Preferably, the stabilizing elements have little tensile elasticity and preferably also little compressive elasticity in the longitudinal direction.

The stabilizing elements of the stiffening layer are preferably formed to a higher degree along the longitudinal direction, so that the angle of the stabilizing elements between the longitudinal axis of the tubing apparatus and the stabilizing element is formed in a range between −45° and 45°, in particular between −30° and 30°. It is also possible to provide different orientations, i.e. two or more different angles to the longitudinal axis of the stabilizing elements with respect to the longitudinal axis. This has the advantage that the bendability is only slightly restricted, but the form fit is strengthened during stiffening in the longitudinal direction between the stiffening layer and the outer tube and preferably the pressure tube.

Preferably, the stabilizing elements are shaped to have high friction with the outer tube due to high pressure in the direction of the tubing apparatus and can lead to stiffening of the tubing apparatus.

The stiffening layer and/or stabilizing elements preferably comprise metals and/or plastics. Stainless steel or polyamide are particularly suitable, for example.

This cohesion between the individual stabilizing elements of the stiffening layer can be formed by mechanical adhesion, such as positive interlocking in recesses, and/or by specific adhesion.

In this context, it would also be conceivable to use stabilizing elements on the outer tube or to use additional stabilizing elements on the inside of the outer tube, which could contribute to stiffening, for example by means of positive locking.

The stabilizing elements of the tubing apparatus are preferably made of metal and/or plastic.

In particular, a plurality of stabilizing elements is preferably interlaced to form a braided structure.

Metals and plastics have very good elastic properties and are usually inexpensive. In addition, good elasticity is very important in the material, so that it can return to its original shape when the applied force is removed by the pressure. This ensures that the tubing apparatus can be stiffened, but can also become elastically deformable again when the pressure is reduced. It is also advantageous if there is no strong elastic hysteresis of the material, i.e. a deformation can remain after the deflecting forces have been removed. Elastic hysteresis is counteracted by the structure of a braided structure, use of an elastic material and/or non-plastic deformation.

The elasticity of the stiffening layer can also be achieved by the type of stiffening layer instead of or in addition to the material elasticity. A relatively loose braided structure can achieve a certain elastic radial deformation by applying a pressure, even if the material of the braided structure is not or hardly elastic per se.

The use of stabilizing elements made of metal and/or plastic also permits cost-effective manufacture. A braided structure can be manufactured very quickly by machine, thus reducing the cost of the invention.

A particularly advantageous design results for the rather acute angles described above, in combination with a braided structure.

Nevertheless, other manufacturing options for creating an advantageous structure of the stabilizing elements, such as laser cutting, would also be conceivable.

The inner tube and the outer tube of the tubing apparatus are preferably designed to be pressure-tight and, in particular, are made of a plastic and/or metal.

The inner and outer tubes preferably both have a wall thickness of in a range of 0.01 to 0.8 mm, in particular 0.2 mm, and may comprise materials such as stainless steel, platinum, iridium, barium, tungsten and/or plastics, in particular thermoplastics such as silicones, polyetheretherketone, fluoropolymers, polyvinylchloride and polyurethane.

When selecting the material, care must be taken to ensure that both the mechanical properties, in terms of flexibility, the surface structure, in terms of thrombogenicity, and the chemistry of the surface, in terms of coatability and charge, are advantageous. The above materials exhibit these advantageous properties and are thus suitable for use in the tubing apparatus.

Preferably, the inner and outer tubes are also reinforced so that the tubes do not twist axially, the cross-section is not so greatly enlarged and/or compressed under pressure, and the tube has higher pressure resistance. For the reinforcement, the inner and outer tubes can be stabilized by spirals, meshes, a wire or a braided structure with transverse orientation. The reinforcement structure is preferably optimized for a ratio of resistance to radial expansion and longitudinal expansion, for example, by adjusting the thickness of the reinforcement and the angle to the tubing apparatus.

The choice of these materials and an advantageous reinforcement for the inner tube and outer tube ensure that the tubing apparatus is elastically deformable but cannot be compressed when used properly. Thus, it is always guaranteed that the inner space or lumina are free, but the tubing apparatus can elastically adapt to the course of the vessels. In the longitudinal direction, the outer and inner tubes should also preferably not be elastically deformable, or hardly so.

The tubing apparatus may include a torsionally rigid structure, preferably a hypotube, helical and/or twisted structure.

The hypotube may be manufactured using process technologies such as laser cutting or core wire welding.

The torsionally stiff structure may be connected to the outer tube, stiffening layer, or inner tube, or may form all or part of the outer tube, stiffening layer, or inner tube.

The tubing apparatus preferably comprises a fluid coupling through which a fluid can be introduced into the region between the inner tube and the stiffening layer, in particular into the pressure tube.

The fluid coupling of the tubing apparatus is advantageous because it allows fluid to be introduced into the area between the inner tube and the stiffening layer. The fluid can be a liquid, for example water, salt water or even a gas. Thus, introducing a fluid between the inner tube and the stiffening layer, particularly into the pressure tube, facilitates stiffening of the tubing apparatus. However, it is preferable to use a liquid, as this is less dangerous to humans when escaping than a gas, in particular air.

The tubing apparatus may preferably comprise at least in part a material visible in X-rays, in particular metal.

In minimally invasive operations with tubing apparatuses, the position of the tubing apparatus and in particular of the inserted end of the tubing apparatus may not be visible to the naked eye. Accordingly, an imaging technique must preferably be used to visualize the inserted tubing apparatus. Preferably, an X-ray method such as fluoroscopy is used so that real-time imaging of the inserted tubing apparatus can preferably be ensured.

Since the attenuation of X-rays is given by the mass attenuation coefficient, a material with elements of a high atomic number should be chosen.

Since plastic often consists mainly of carbon compounds that absorb X-rays poorly, the tubing apparatus should instead contain visible material such as modified plastic or metals. Even small amounts of iron, for example, can ensure the visibility of the tubing apparatus.

A front portion of the tubing apparatus may preferably be tapered to prevent the formation of a step.

According to the invention, the anterior portion of the tubing apparatus is the portion that is first inserted into the patient's vessel.

The front area of the tubing apparatus should be as conical as possible, as this offers the advantage of minimizing the risk of injury when using the tubing apparatus. When inserting the tubing apparatus into a patient, a conical front area of the tubing apparatus causes less or no injury and pain. In addition, the force required is less and the application is easier to perform. Also, when guiding along the vessels of a human, a conical anterior region is advantageous because, firstly, the curves in the vessels are easier to perform when it is a narrower anterior region, which is also thus easier to elastically deform. Secondly, a conical anterior region minimizes injury to vessels and the potential loosening of deposits or clots.

A hydrophilic or hydrophobic coating can be arranged on the outside of the outer tube of the tubing apparatus. In addition, a coating in the inner tube with polytetrafluoroethylene is particularly advantageous.

Among other things, the coating of the outer tube can improve sliding behavior and minimize friction. On the other hand, biocompatibility must be ensured or the risk of infection kept low, and hemocompatibility, antithrombogenicity or low particle release optimized. Coatings using polymers are particularly suitable for this purpose.

In the coating processes, the surface is preferably activated beforehand to be able to accept coatings. This can be done, for example, by plasma activation, in which an electron plasma causes open bonding sites to form on the surface, which are occupied by polar hydroxy groups. This creates a hydrophilic surface and the polymer coating can adhere better.

The surface to be coated can be made hydrophilic or hydrophobic, depending on the precursor monomers, for example, by plasma polymerization in a vacuum by feeding gas with precursor monomers deposited on the surface. Other coating processes such as dip coating, spray coating, reel-to-reel coating are also conceivable.

In a final step, the molecules of the coating can be further crosslinked by applying thermal energy or UV radiation.

Polytetrafluoroethylene is suitable as a hydrophobic coating for the outer tube, since the molecule is nonpolar due to its symmetrical structure, has very low surface tension, and exhibits low cohesion and adhesion forces to other substances. Other polyfluoroethylene, hexafluoropropene and/or polydimethylsiloxane are also conceivable as hydrophobic coatings, with polydimethylsiloxane even having an advantageous antithrombogenic effect.

Suitable hydrophilic polymers, on the other hand, are coatings that can form hydrogen bonds so that a film of water can be easily bound to the surface. Hydrophilic coatings can consist of only one hydrophilic polymer, or comprise a combination of hydrophilic polymers.

Advantageous hydrophilic coatings that can be imagined include hydrogels or polyvinylpyrrolidone, the properties of which can be further adapted by adding vinylpyrrolidone copolymers.

The stiffening section of the tubing apparatus may form part or all of the tubing apparatus.

The length and area of the stiffening section can advantageously be easily adapted to the particular problems. A length of the stiffening layer of around 30 cm to the front end, or complete stiffening of the tubing apparatus, is particularly advantageous.

Thus, the stiffening section can occupy only the area needed to hold the tubing apparatus in place in one area. The rest of the tubing apparatus can benefit from the advantages of not having a stiffening layer, such as increased elasticity. Thus, costs can also be reduced while still ensuring complete functionality.

The overall length of the tubing apparatus is preferably between and 120 cm, depending on the desired use, and the length of the stiffening section may extend over the entire length, or a section.

The inner tube of the tubing apparatus can have an inner diameter of 1 to 11.33 mm.

A small outer diameter is very important for minimally invasive procedures. Nevertheless, a certain minimum inner diameter is necessary for the passage of wires, tools and/or stents. The inner diameter of catheters is expressed in French, where one French corresponds to 0.33 mm. Thus, the tubing apparatus has an inner diameter of 3-32 Fr. In order to achieve a small inner diameter, allowing the insertion of tools and, for example, stents, and to ensure the stiffness of the tubing apparatus, the tubing device layers must also be produced with very thin walls. A small inner diameter is necessary to fit into narrow vessels, or to be able to complete turns of up to about 180° with the tubing apparatus in vessels. Therefore, it is a particularly advantageous feature of the invention that the tubing apparatus is fully functional even with a small inner diameter and can be manufactured at very low cost. The outer diameter is preferably at most 4 Fr, in particular at most 2 Fr, larger than the inner diameter. Thus, with an inner diameter of 6 Fr, the outer diameter is at most 10 Fr, so that the tubing apparatus is very compact and can be more easily guided around branches of vessels.

Preferably, the inner tube of the tubing apparatus can have at least two lumens so that multiple devices can be inserted through the tubing apparatus in separate lumens.

Multiple separate lumens in a tubing apparatus can be advantageous so that when tools and/or equipment are used simultaneously, snagging or jamming cannot easily take place. This offers the advantage that several functions, such as guiding the guide wire and inserting, and/or preparing tools and/or devices can be performed simultaneously. Furthermore, a shaped wire can thus be used in parallel with a guide wire in one lumen at a time. Preferably, an additional soft dilator is nevertheless used.

The set comprises at least one tubing apparatus as previously described and a dilator, in particular at least one soft dilator and a shaped wire and/or guide wire.

Thus, the full range of functions is guaranteed and all components are compatible.

The wire can be a guide wire and/or a shaped wire.

The guide wire and the shaped wire can be inserted into the inner tube. In particular, the shaped wire has bends for guiding the tubing apparatus and/or is deformable into bends. Preferably, a bend of the shaped wire can be adapted so that the shaped wire can be used flexibly, as known from the prior art.

In particular, a shaped wire is rotationally stable and/or torsionally stable to guide the dilator tip.

The shaped wire and/or the lumen into which it is insertable are preferably coated, treated, or formed from a very low friction material so that the shaped wire can be inserted and removed as easily as possible.

Common shapes for shaped wire are straight wire, field hockey stick, cobra, SOS and Berenstein. The shaped wire can have one or two shaped ends.

In addition, the set preferably comprises at least one dilator. In particular, the dilator is designed to be easily deformable, and thus a soft dilator.

The soft dilator can also be called an adapter.

The shaped wire is used so that the flexible front section of the tubing apparatus and/or the dilator can be controlled as a shaping device in the vessels. For this purpose, the shaped wire is preferably bent in accordance with the intended use, and can be inserted into the tubing apparatus. The bend of the shaped wire is preferably very flexible, so that when it is inserted, a bend is only given by the shaped wire at the end of the tubing apparatus and/or at the dilator, since the stiffness of the shaped wire is only sufficient to bend it at the end of the tubing apparatus and/or at the dilator. Preferably, the tubing apparatus is not bendable by the shaped wire, but only the soft dilator. The bending can thus be completed by the shaped wire and the tubing apparatus can then be further inserted or stiffened in the desired vessel.

The soft dilator is preferably longer than the respective tubing apparatus, in particular at least 15 cm longer.

The soft dilator may be soft along its entire length, or only at the tip of the soft dilator.

When the soft dilator is fixed with its longitudinal axis horizontal so that the anteriormost 5 cm to the tapered tip of the dilator are free, and a weight of 30 g is applied orthogonally to the tip of the dilator, the tip of the soft dilator deflects by a range of 30 mm to 48 mm, especially 42 mm. In comparison, the tip of a hard dilator deflects by only 23 mm. The deflection of a soft dilator is thus essentially at least 25% greater compared to a hard dilator, with the same shape of dilators.

The taper of the soft dilator is 20 to 80 mm, especially 40 mm, long from the tip, at the opening for the guide wire, to the widening to the inner diameter of the tubing apparatus.

The soft dilator can have two lumens, one lumen for the guide wire and one lumen for the shaped wire. The lumen for the shaped wire is closed in the tip of the dilator, the lumen for the guide wire is open.

A closed lumen for the shaped wire has the advantage that the shaped wire can only be used to form the shape and cannot damage the vessels. In addition, the shaped wire is preferably not tapered, but flattened, so that the interior of the lumen, as well as the closed end, cannot be damaged.

Another lumen also offers the advantage that a lumen for introducing substances can be used in parallel with the moldability of the dilator and/or the anterior tubing apparatus through the shaped wire. Thus, for example, a contrast agent can be introduced into the vessel without having to remove the shaped wire.

The inner diameter of a lumen of the dilator with respect to the application of a guide wire is preferably in a range of substantially 0.2 mm to 1.2 mm, in particular 0.35 mm to 0.97 mm.

The transition between the tubing apparatus and the dilator preferably closes substantially positively and the dilator preferably has an outer diameter of substantially 1 mm to 11.33 mm in this region.

The soft dilator may be hydrophilically coated, again by the measures outlined above with respect to the tubing apparatus. The soft dilator is preferably softer than the shaped wire so that the dilator can be deformed by the shaped wire.

Preferably, the soft dilator is visible in the X-ray, in particular by the measures already outlined with regard to the tubing apparatus.

In particular, at least the anteriormost 4 cm of the dilator shall be located by radiographic techniques.

The soft dilator can also be used independently of the tubing apparatus described above with previous tubing apparatuses.

The soft dilator can be made of silicone or thermoplastics.

The soft dilator can be identified by color marking from conventional hard dilators, or dilators included in the set.

The hard dilator is inserted as the first part before the tubing apparatus after insertion of the guide wire into the vessel and temporarily dilates the vessel for insertion of the tubing apparatus. The dilator is conically shaped for this purpose.

The set can additionally include a hard dilator. Thus, the physician can choose which dilator fits better in the situation.

Further leading to the solution of the task is a method for treating a patient, wherein a tubing apparatus as previously described is inserted into a vessel of a patient.

Such a procedure leads to a safe and fast treatment of the patient.

The tubing apparatus can be stiffened once the tubing apparatus is positioned on a target vessel.

This provides safe access for the actual treatment of the target vessel.

As has been the practice in the past, a needle is preferably first used to create access into an access vessel. A guide wire is inserted through this needle into the access vessel and the needle is then removed from the vessel. A dilator, in particular a hard dilator, is inserted into the access vessel via the access wire. The hard dilator is thus preferably used and sold together with a tubing apparatus and is known in the prior art. A hard dilator is insertable into the tubing apparatus so as to surround the guide wire and occupy the area between the guide wire and the tubing apparatus. The hard dilator is very stiff and thus can be used to expand the vessel wall as it enters the vascular system.

When the hard dilator is fixed with its longitudinal axis horizontal so that the anteriormost 5 cm are free to the tapered tip of the dilator, and a weight of 30 g is applied orthogonally to the tip of the dilator, the tip of the hard dilator deflects by a range of 10 to 35 mm, especially 23 mm. The taper of the hard dilator is 20 to 80 mm, in particular 40 mm, long from the tip at the opening for the guide wire to the widening to the inner diameter of the tubing apparatus.

The hard dilator can, preferably subsequently, be exchanged for a soft dilator. The soft dilator can then be advanced to the front of the target vessel. The guide wire can then be advanced into the target vessel. The guide wire and shaped wire are then fixed and the dilator with tubing apparatus is advanced into the target vessel and fixed. The tubing apparatus is then also advanced into the target vessel in a flexible state and stiffened.

After the tubing apparatus has been stiffened, the soft dilator with the shaped wire is pulled out.

The desired intervention in the target vessel can then begin.

This results in the advantage that a soft dilator can be used simultaneously as an optimized guiding device and diagnostically. Thus, additional time can be saved and the procedure can be performed technically easier without changing the material.

The problem is further solved by a method for selectively stiffening a tubing apparatus.

By introducing a fluid into the space between the inner tube and the stiffening layer, the tubing apparatus is stiffened.

Selective stiffening of the tubing apparatus provides both the flexibility needed for insertion into vessels and the selective stability of a stiffened tubing apparatus or stiffening section. Time of use is also minimized, as a prior art tubing apparatus can often be deflected from its site of use, for example, by blood flow, manipulation within the tubing apparatus, or slippage of the tubing apparatus.

Furthermore, for safety reasons, a fluid should preferably be selected in such a way that even in the event of severe damage to the tubing apparatus, no damage to health can occur. In this context, it would also be conceivable to have a device for monitoring the pressure exerted by the fluid introduced.

By reducing the pressure in the space between the inner tube and the stiffening layer, the tubing apparatus can become flexible again. Preferably, the hose is in the elastic state when reduced to essentially atmospheric pressure and pressure in the blood vessels of a human.

It is particularly important that this process can be selectively activated and deactivated. Thus, the tubing apparatus can be inserted flexibly. At the target site, the tubing apparatus can be stiffened for the minimally invasive surgical procedure, according to individual anatomy, and then made elastic again for removal to avoid possible damage to health. Possible damage, such as strokes, can be caused by the loosening of debris or thrombi, as well as inserted tools and/or devices and/or small parts. An at least partially stiffened tubing apparatus minimizes these risks and enables more precise work.

It is therefore of particular importance to avoid this potential damage and a great advantage of the invention that selective stiffening of the tubing apparatus can be accomplished as needed by introducing a fluid into the space between the inner tube and the stiffening layer.

It is also advantageous that pressure only needs to be built up to stiffen the tubing apparatus and released to make it elastic, as this ensures that the tubing apparatus can be released even if the pressure device fails. If pressure had to be built up to make it elastic, in the case of a leaking tubing apparatus, a stiffened tubing apparatus would be in a person's vessels. This would not be removable, or only with great difficulty and/or at great risk to the person.

In the following, embodiments of the invention are described in detail with reference signs. Here shows

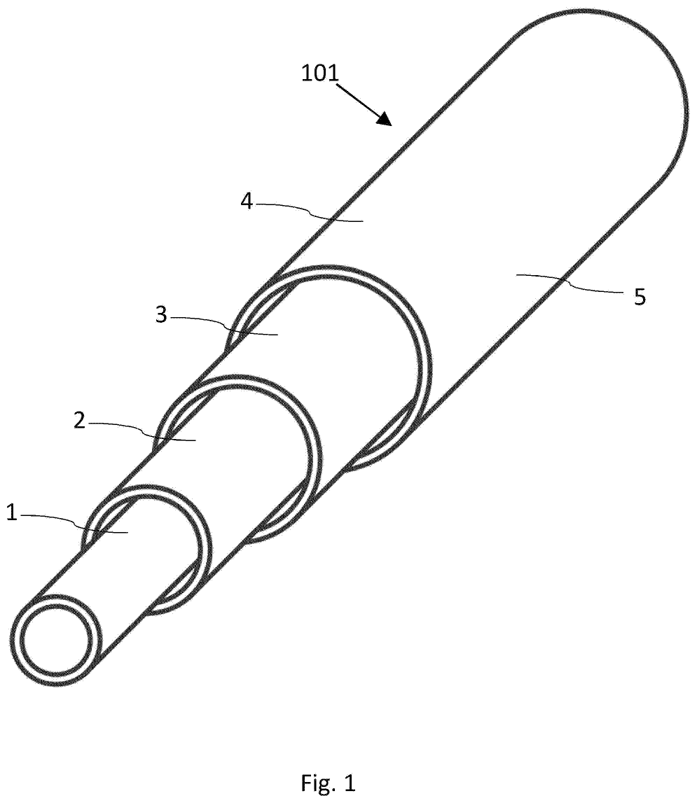

FIG. 1: a stiffening section of the tubing apparatus,

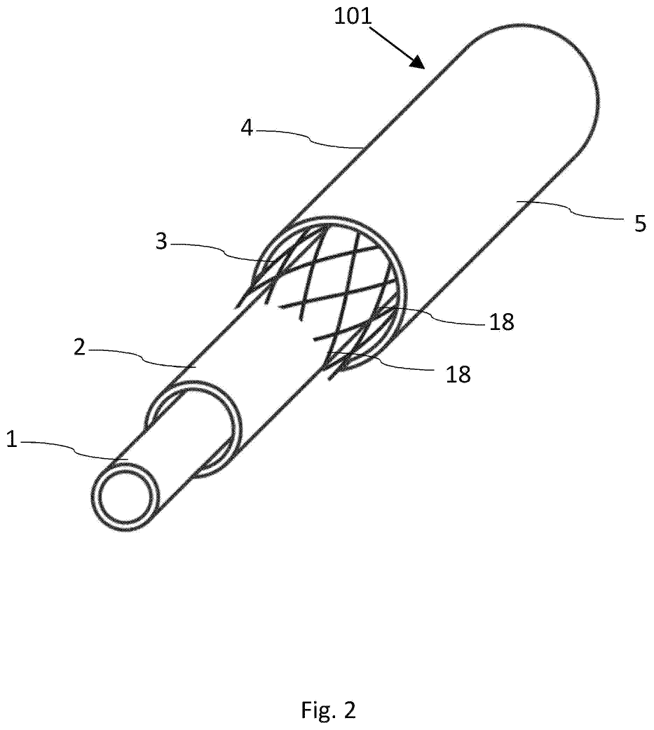

FIG. 2: an embodiment of the stiffening section,

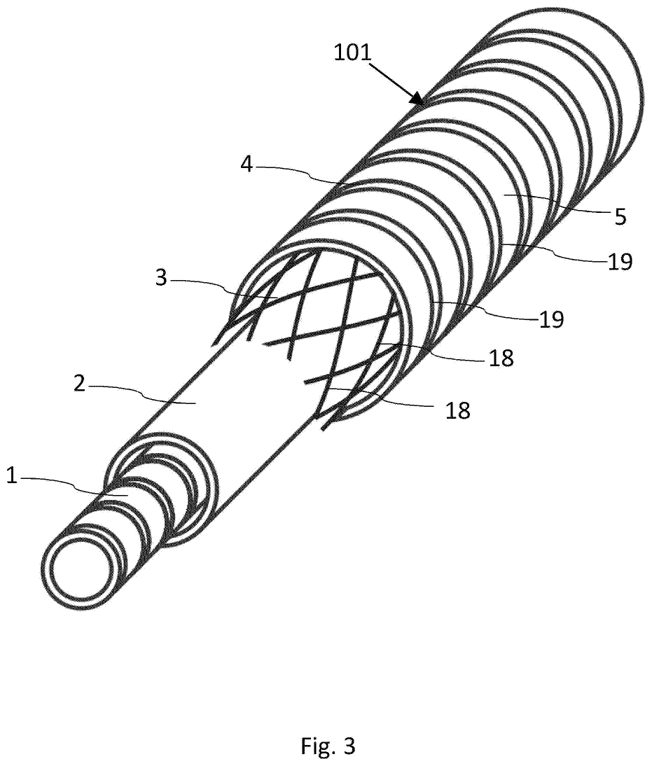

FIG. 3: an embodiment of the stiffening section,

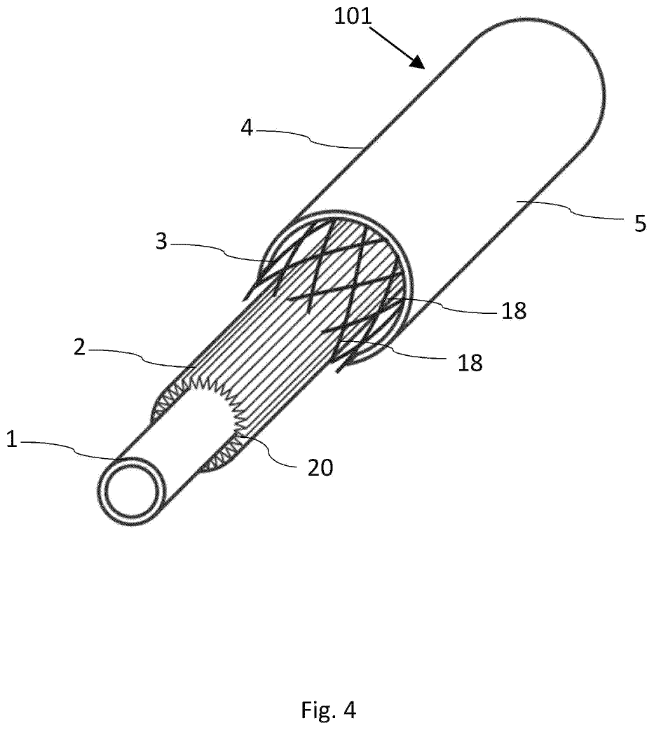

FIG. 4: an embodiment of the stiffening section,

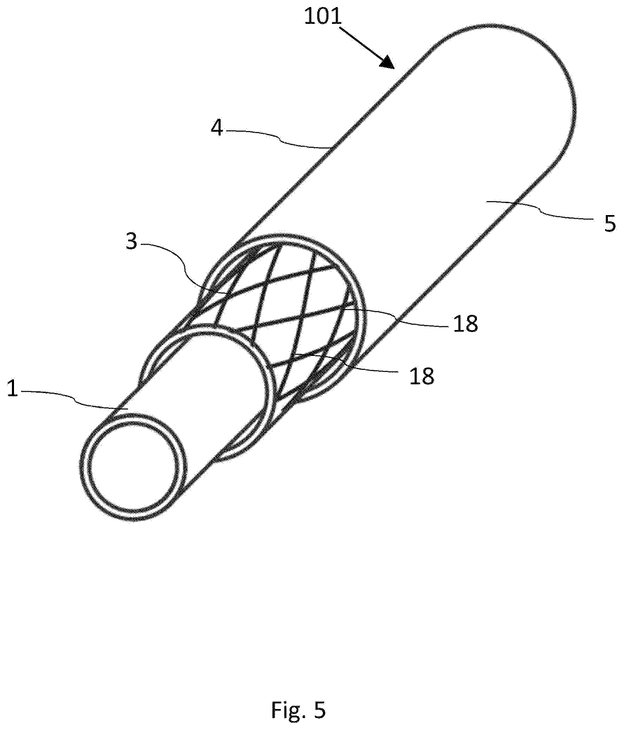

FIG. 5: an embodiment of the stiffening section,

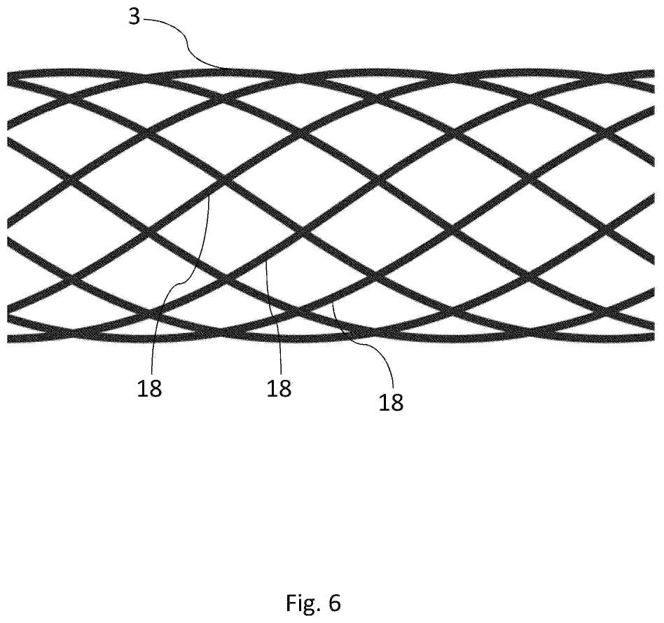

FIG. 6: a structure of the stiffening layer,

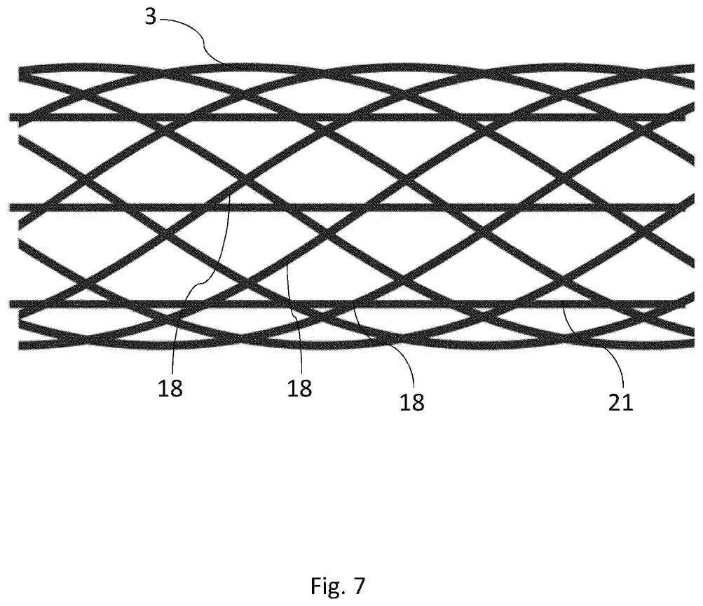

FIG. 7: an alternative structure of the stiffening layer,

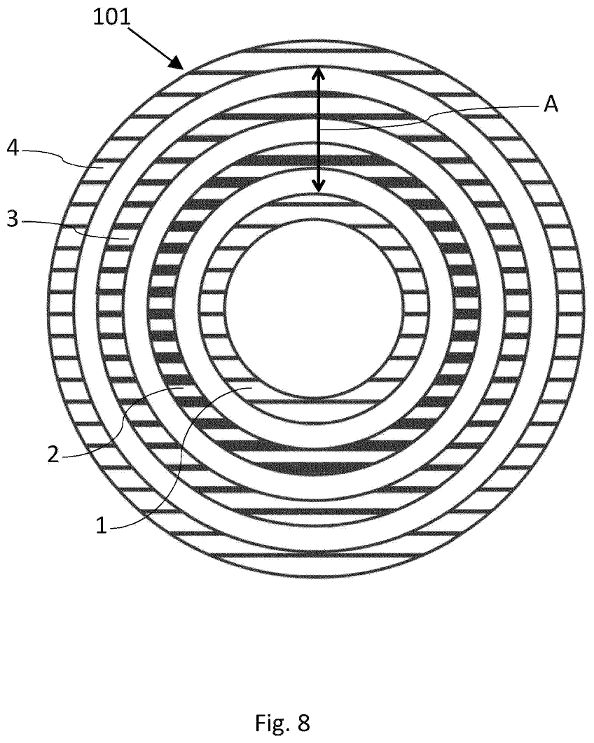

FIG. 8: a cross-section of the stiffening section of the tubing apparatus,

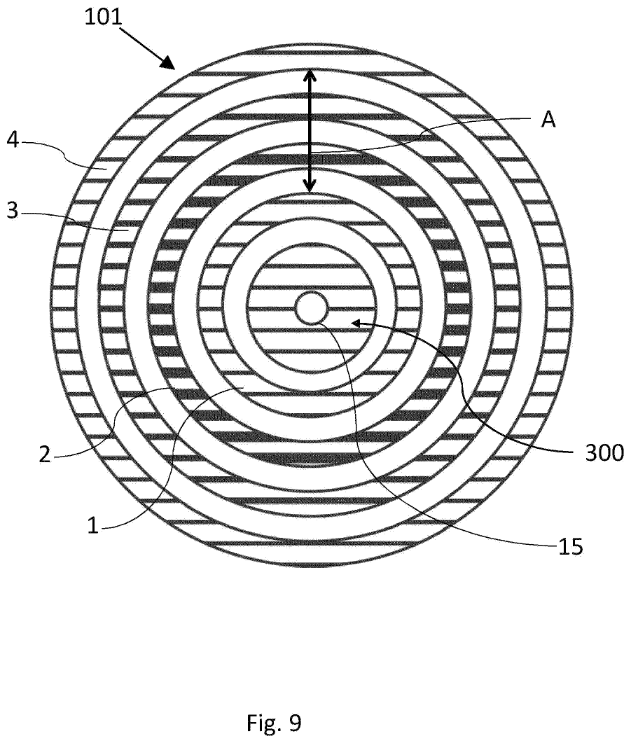

FIG. 9: a cross-section of the stiffening section with the dilator inserted with a lumen,

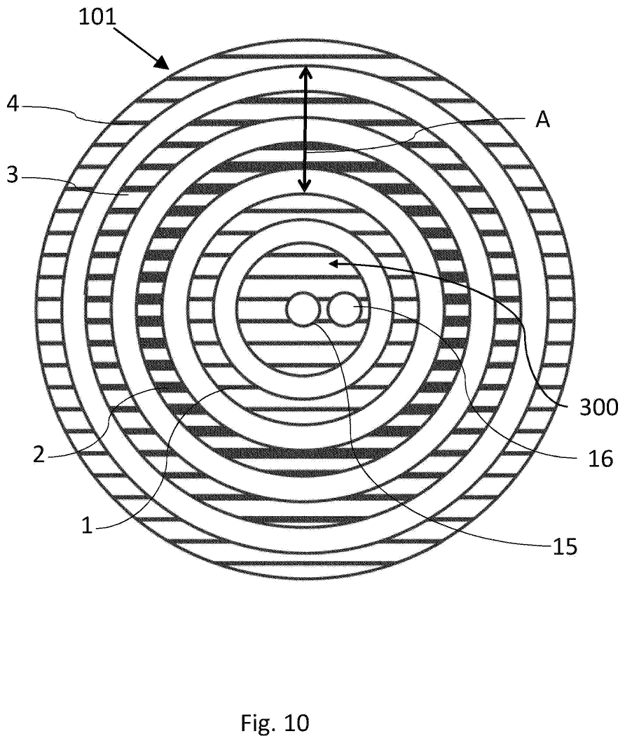

FIG. 10: a cross-section of the stiffening section with the dilator inserted with two lumens,

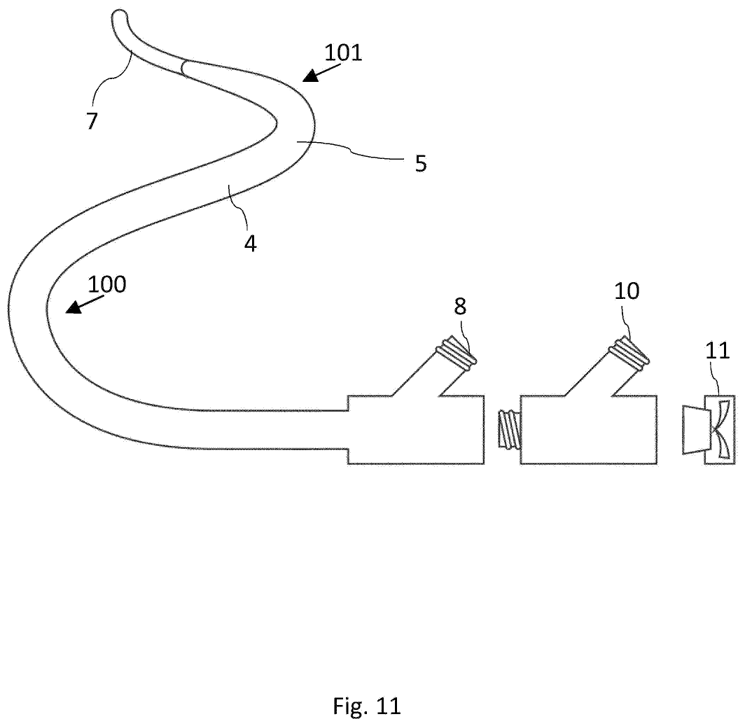

FIG. 11: a tubing apparatus with stiffening section,

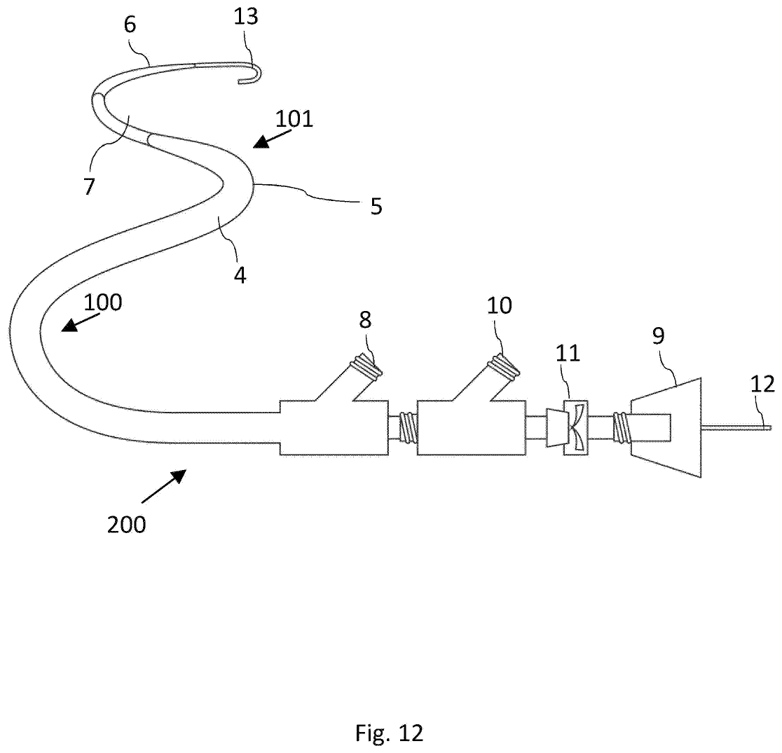

FIG. 12: a set of tubing apparatus with dilator and additional guide wire,

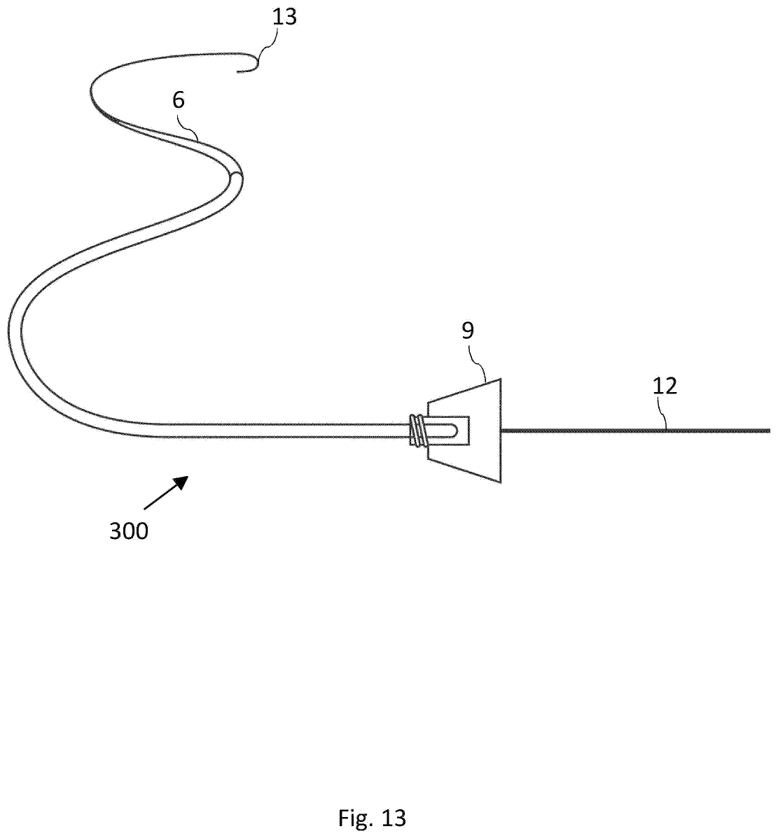

FIG. 13: a dilator with an inserted guide wire,

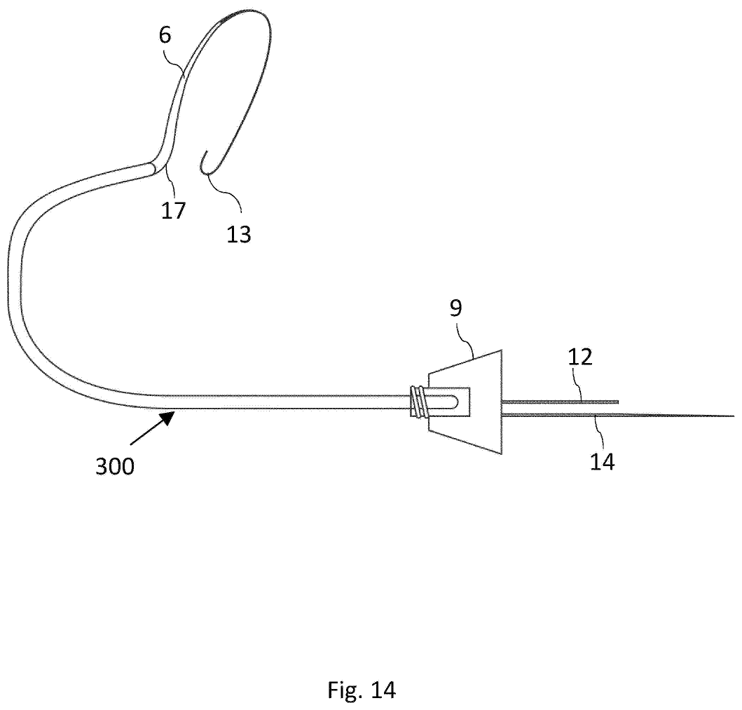

FIG. 14: a dilator with an inserted guide wire and shaped wire,

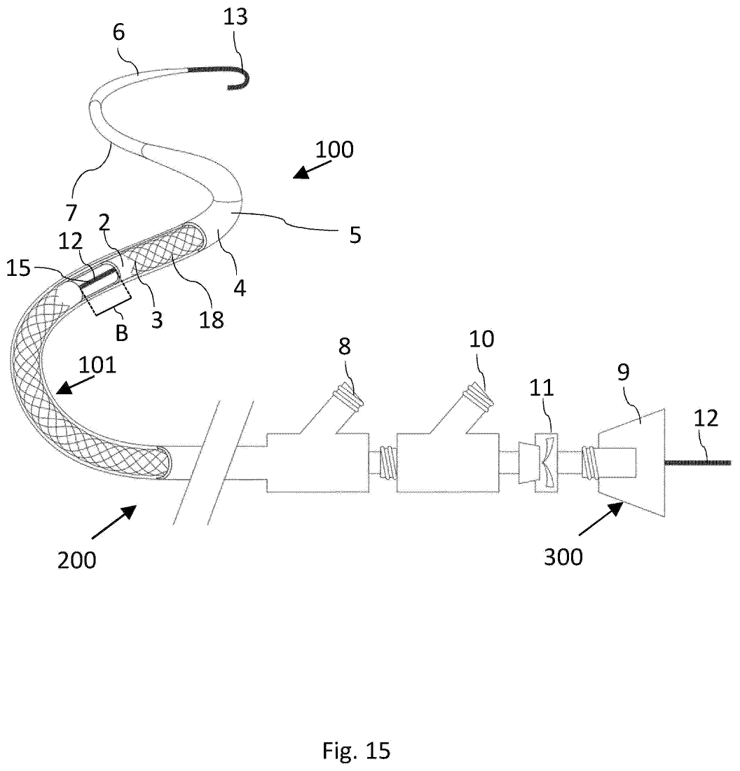

FIG. 15: a set of tubing apparatus with dilator and additional guide wire with partially removed layers,

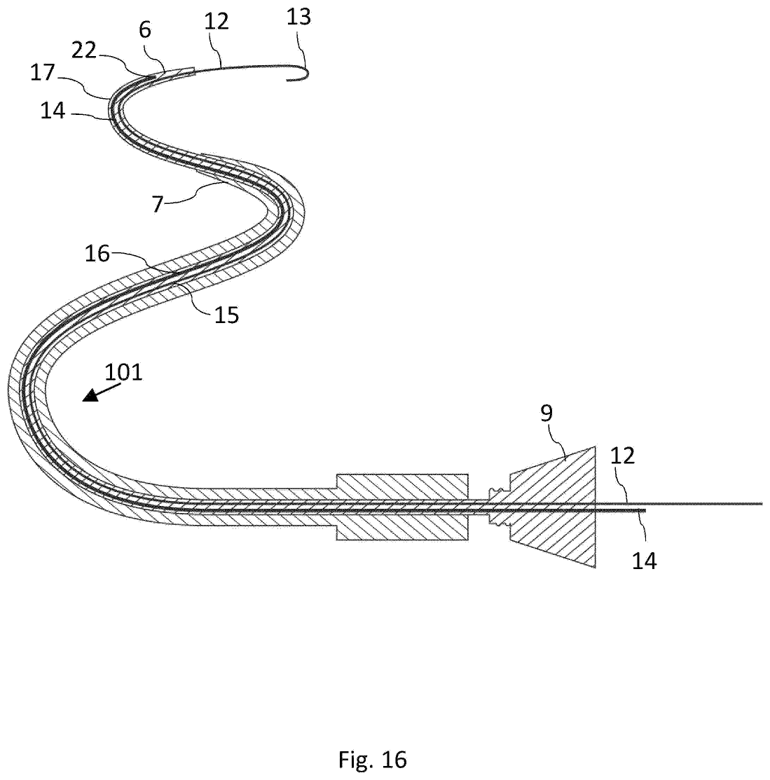

FIG. 16: a longitudinal section of a dilator in a tubing apparatus with two lumens,

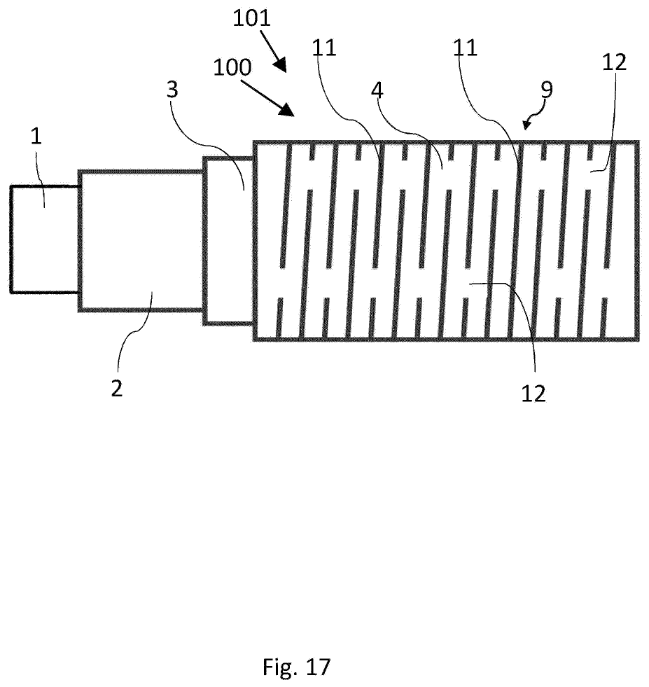

FIG. 17: a first embodiment of a tubing apparatus with a torsionally rigid structure in profile,

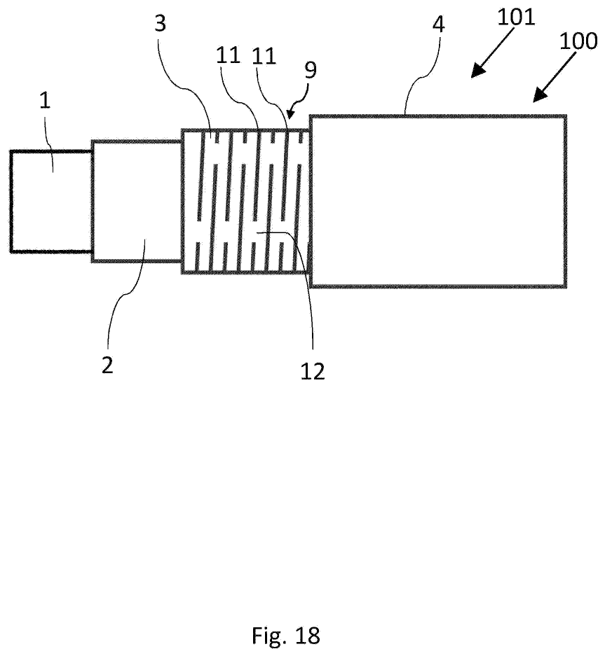

FIG. 18: a second embodiment of a tubing apparatus with a torsionally rigid structure in profile,

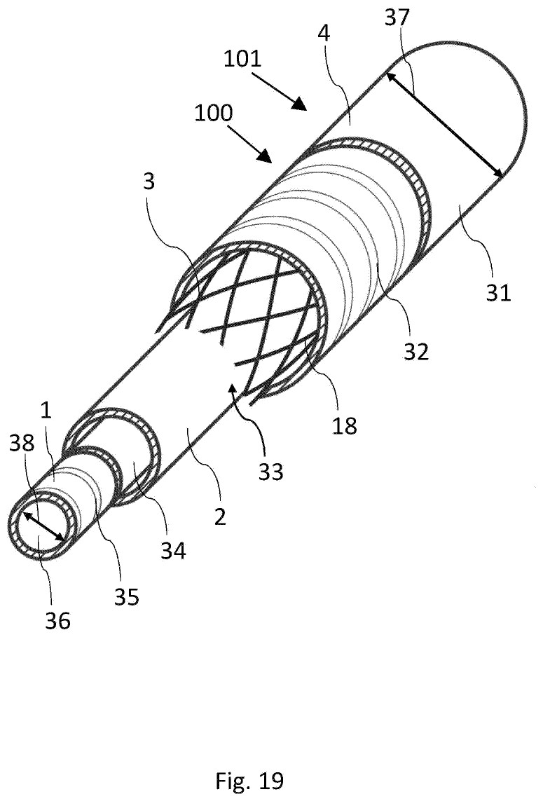

FIG. 19: a perspective view of an embodiment of a tubing apparatus having a stiffening section.

Identical reference signs in the figure show identical components.

FIG. 1 shows a stiffening section 101 of the tubing apparatus 100 of a preferred embodiment of the invention having four layers of tubing. The tubing apparatus 100 is shown in open sections of the individual layers for better visibility. From the outside to the inside, there is an outer tube 4, a stiffening layer 3, a pressure tube 2, and an inner tube 1.

The pressure tube 2 is filled with an isotonic sodium chloride solution at a pressure of 16 bar for stiffening, and thus the stiffening layer 3 presses against the outer tube 4 by moving radially outward. When the pressure is removed, the stiffening layer 3 is also moved radially inwards again and the stiffening of the stiffening section 101 decreases.

In this embodiment, the pressure tube 2 is made of thermoplastics and can thus be expanded and elastically radially deformed by the introduction of a saline solution.

The stiffening layer 3 can also be moved radially inwards again when the applied pressure is removed. This ensures that the tubing apparatus 100 can always be removed, especially when the pressure can no longer be built up. The tubing apparatus 100 is designed to be movable in its basic state without pressure, so that there is no danger from irreversible stiffening of the tubing apparatus 100 in the event of a defect. In this case, the stiffening layer 3 is formed of a loose braided structure of stainless steel and/or plastic, which is movable with respect to each other, and which extends to a partial vector in the longitudinal direction. Thus, the stiffening layer 3 can easily expand and a strong friction with the outer tube can be established.

In this embodiment, the outer tube 4 and inner tube 1 comprise polysiloxane and a stainless steel spiral, the stainless steel spiral being helically oriented along the longitudinal axis of the tubing apparatus 100 and being embedded in the polysiloxane and completely enclosed. In this context, however, the alternative use of polyurethane for the inner 1 and outer tubes 4 would also be conceivable. The inner surface of the inner tube 1 is coated with polytetrafluoroethylene to provide the lowest possible frictional resistance within the inner tube for inserted equipment, tools, liquids and small parts. The outer tube 4 is also hydrophilic due to a coating 5 with polyvinylpyrrolidone. Thus, insertion into a body passage is easier and can be performed atraumatically. Furthermore, the coating 5 increases the sliding properties of the tubing apparatus 100 within the vessels.

FIGS. 2-5 show explicit embodiments alternatively to FIG. 1. For analogous reference signs, functions and embodiments of the invention that are not explicitly mentioned, reference is made to FIG. 1.

FIG. 2 shows the stiffening section 101 of the tubing apparatus 100, which shows a stiffening layer 3 with a braided structure made of stainless steel, the individual stabilizing elements 18 of the stiffening layer 3 being manufactured so as to be displaceable and/or slidable relative to one another. The stiffening layer 3 can be moved radially outwardly by the pressure tube 2 without strong resistance, and can stiffen the stiffening section 101 by friction with the outer tube 4.

However, other materials for the stabilizing elements 18, such as polyamide, are also conceivable.

FIG. 3 shows an embodiment of the stiffening section 101 of the tubing apparatus 100 analogous to FIG. 2. In addition to the braided structure of stainless steel of the stiffening layer 3 with stabilizing elements 18, the reinforcement 19 of stainless steel of the inner and outer tubes 1, 4 has been shown.

The reinforcement 19 is formed as concentrically as possible around the longitudinal axis of the tubing apparatus 100, so that the tubing apparatus 100 continues to be formed as flexibly as possible to the sides when it is not in the stiffened state. In this embodiment example, the reinforcement 19 is realized by rings, but a spiral-shaped reinforcement 19 and a mesh-shaped reinforcement 19 are also conceivable.

FIG. 4 shows an embodiment of the stiffening section 101 of the tubing apparatus 100, wherein the pressure tube 2 can press radially outward against the outer tube 4 by unfolding a folded structure 20, the stiffening layer 3 and stabilizing elements 18. Thus, the expansion of the pressure tube 2 does not change even after repeated use, since there is no or essentially no elastic deformation. The folded structure 20 of the pressure tube 2 unfolds as soon as pressure is applied with the pressure tube 2 by introducing the salt solution, and folds back in when the pressure is removed. This also has the advantage that elastic hysteresis is minimized by the folded structure 20. Thus, it minimizes the persistence of deformation after the deflecting force is removed. Thus, the safety, as well as the durability of the pressure tube 2 of the tubing apparatus 100 is ensured.

FIG. 5 shows an embodiment of the stiffening section 101 of the tubing apparatus 100, wherein the stiffening layer 3 forms a dense tube as a unit with a plastic and the stabilizing elements 18. The stiffening layer 3 can thus additionally assume the function of the pressure tube 2 and be pressed radially outwardly against the outer tube 4 by an applied pressure to stiffen the stiffening section 101.

FIG. 6 shows an embodiment of the stiffening layer 3 with stabilizing elements 18, wherein the braided structure formed is not rigidly connected to one another at the interfaces, but is designed to be freely movable relative to one another in order to ensure radial deformability. In this embodiment example, the stabilizing elements 18 are formed at an angle of substantially 30° to the longitudinal axis of the tubing apparatus 100.

FIG. 7 shows another embodiment of the stiffening layer 3 with stabilizing elements 18 analogous to FIG. 6, with additional longitudinal stabilizing elements 21 essentially parallel to the longitudinal axis being shown, which additionally increase friction when pressed radially against the outer tube 4.

FIG. 8 shows a cross-section of the stiffening section 101 of the tubing apparatus 100 with four tubing layers. From the outside to the inside, an outer tube 4, a stiffening layer 3, a pressure tube 2 and an inner tube 1 can be seen. In addition, the area A between the inner tube and the outer tube 4, between which the pressure can be built up, has been marked. Due to the applied pressure between the inner tube 1 and the pressure tube 2, a radial movement of the stiffening layer 3 outward through the pressure tube 2 can be executed, which presses the stiffening layer 3 onto the outer tube 4 and thus stiffens it. The choice of materials and mode of operation is otherwise analogous to FIG. 1.

FIG. 9 shows the cross-section of the stiffening section 101 with the four hose layers 1, 2, 3, 4 analogous to FIG. 8. In addition, FIG. 9 shows an inserted soft dilator 300, which has a lumen 15 for the guide wire 12, as well as for the introduction and removal of substances and/or tools from/to the vessels. In the area A between the inner and outer tubes 1, 4, radial displacement of the pressure tube 2 and the stiffening layer 3 can take place by pressure to stiffen the stiffening section 101. During the stiffening process, the pressure in the space between the inner tube 1 and the pressure tube 2 is increased to such an extent that the pressure tube 2 moves radially and presses the stiffening layer 3 against the outer tube 4.

FIG. 10 shows the cross-section of the stiffening section 101 analogous to FIG. 9, wherein the inserted soft dilator 300 has the lumen 15 for the guide wire 12, as well as a lumen 16 for the shaped wire 14. The lumen 15 for the guide wire 12 is further centered in the soft dilator 300, while the lumen 16 for the shaped wire 14 is offset to one side. Guidance through the vessels with the guide wire 12 is thus further ensured by the radially symmetrical shape, while the shaped wire 14 can additionally adapt the shape of the soft dilator 300.

FIG. 11 shows the tubing apparatus 100 having a stiffening section 101. The tubing apparatus 100 shows a tapered front portion 7 so that the tubing apparatus 101 can be used more easily and atraumatically for penetrating vessels. A fluid coupling 8 is shown for supplying fluid and stiffening the stiffening section 101 of the tubing apparatus 100. In addition, a side port 10 is shown for supplying fluid. A hemostatic valve 11 also prevents blood from leaking from the tubing apparatus 100 while still allowing devices to be inserted. The outer tube 4 also has a hydrophilic coating 5.

FIG. 12 shows the set 200 of a tubing apparatus 100 and a stiffening section 101 having a perimeter, preferably at least included in the set.

FIG. 12 shows the guide wire 12 having a curved front end 13 for guiding the tubing apparatus 100. Furthermore, a conical long tip 6 of a soft dilator 300 formed of very elastic polysiloxane can be seen, which can be used as a guiding device. In addition, the front portion 7 of the tubing apparatus 100 is tapered to facilitate insertion of the tubing apparatus 100 and preferably also to facilitate guiding the tubing apparatus 100 around coils in vessels. This is further facilitated by a hydrophilic coating 5 on the outer tube 4.

Furthermore, a fluid coupling 8 can be seen in FIG. 12, which allows a fluid, preferably a saline solution, to be supplied.

FIG. 12 also shows the guide element 9 of the soft dilator 300, which can be used to control and translate the tip of the dilator 6. A side port 10 also allows fluids to be supplied, such as a contrast solution, while closing the tubing apparatus 100.

Furthermore, the tubing apparatus 100 of the set includes a hemostatic valve 11 that closes the set 200 and allows insertion of devices, tools, and/or small parts, but prevents leakage of blood. In this embodiment, the tubing apparatus 100 and the soft dilator 300 comprise iron dust so that the movements can be visualized by X-ray methods.

FIG. 13 shows the soft dilator 300 with an inserted guide wire 12. The front end of the guide wire 12 has a bend 13. The tip of the dilator 6 is tapered for easy maneuvering and insertion. It should be noted here that the guide wire 12 is movable independently of the soft dilator 300, and thus can provide a path into a vessel. In this regard, the guide element 9 of the soft dilator 300 can be used to move in the longitudinal direction of the soft dilator 300. The soft dilator 300 can thus be pushed along the predetermined direction of the guide wire 12.

FIG. 14 shows the soft dilator 300 with inserted guide wire 12, as well as inserted shaped wire 14. Here, the shaped wire 14 can be inserted laterally into a second separated lumen 16, as shown in FIG. 10, so that the lumen 15 of the guide wire 12 can still be arranged centrally in the dilator 6. The tip of the dilator 6 is controllable by means of the guide element 9 according to the predetermined bends 13 of the guide wire 12, as well as dilator deformation 17 by the shaped wire 14 in the vessels and can be moved and/or stiffened along the directions predetermined by the wires 12, 14.

FIG. 15 shows the set 200 of the tubing apparatus 100 with a stiffening section 101 analogous to FIG. 12, although analogous designations have not been repeated. The outer tube 4 has been partially removed for better visualization, so that the stiffening layer 3 and stabilizing elements 18, as well as the pressure tube 2 underneath, can be seen. In addition, a section B of the stiffening section 101 is shown with the stiffening layer 3 and pressure tube 2 additionally removed so that the guide wire 12 is visible in the lumen 15 of the soft dilator 300.

FIG. 16 shows a soft dilator 300 with a lumen 15 for the guide wire 12 and a lumen 16 for the shaped wire 14. For a better illustration of the embodiment, the additional layers of a tubing apparatus 100 according to the invention with stiffening section 101 have been omitted in this longitudinal section.

The longitudinal section shows that the tip of the dilator 6 protruding from the conical front portion 7 of the tubing apparatus 100. The tip of the dilator 6 is formed into a dilator deformation 17 by the shaped wire 14. The shaped wire 14 cannot exert enough force to shape the tubing apparatus as it is inserted and removed, but rather the deformation into a dilator deformation 17 occurs only at the tip of the dilator 6. In addition, FIG. 16 shows that the lumen 16 for the shaped wire 14 has a terminated end 22. Thus, the shaped wire 14 can provide a dilator deformation 17 that is present even when the guide wire 12 must be removed. Further, the dilator deformation 17 of the shaped wire 14 in combination with the predetermined bend 13 of the guide wire 12 can provide a practicable guide for the tubing apparatus 101 in vessels.

FIG. 17 shows a first embodiment of a tubing apparatus 100 having a torsionally rigid structure 9. A stiffening section 101 of the tubing apparatus 100 comprises, from the inside to the outside, an inner tube 1, a pressure tube 2, a stiffening layer 3 and an outer tube 4. The tubing apparatus 100 is shown in profile in open sections of the individual layers for better visibility.

The outer tube 4 comprises a hypotube, predominantly of metal, which has helical cuts 11 along the longitudinal axis of the tubing apparatus 100.

The outer tube 4 is designed to be essentially immobile radially. The helical cuts 11 along the outer tube 4 form the torsionally rigid structure 9. In this embodiment, the helical cuts 11 are arranged along a direction of rotation about the longitudinal axis.

The helical cuts 11 do not extend continuously along the surface of the outer tube 4, but have material bridges 12. In this embodiment, the helical cuts 11 each extend around the outer tube 4 by nearly one revolution and are arranged alternately with staggered helical cuts 11.

FIG. 18 shows a second embodiment of a tubing apparatus 100 with a torsionally stiff structure 9 formed on the stiffening layer 3. The torsionally stiff structure 9 on the stiffening layer 3 is designed to be radially movable. The choice of materials and operation of the tubing apparatus 100 is otherwise analogous to FIG. 10 and no further description is given.

FIG. 19 shows a perspective view of a tubing apparatus 100 having a stiffening section 101. The tubing apparatus 100 comprises, from the inside to the outside, an inner tube 1, a pressure tube 2, a stiffening layer 3, and an outer tube 4. Layers and tubes and components of the layers and tubes have been at least partially removed to improve visibility.

The outer tube 4 comprises an outer polymer layer 31 with a radial width of 0.08 mm. Beneath the polymer layer 31 is a reinforcement 31 of the outer tube 4, which has a radial width of 0.05 mm. The stiffening layer 3 consists of in a helical braided structure with stabilizing elements 18 which are arranged at an angle to each other, so that at the crossing points in each case two stabilizing elements contact each other and are arranged one above the other in a radial direction. The stabilizing elements each have a radial width of 0.08 mm. In addition, a cavity 33 with a radial width of 0.05 mm is arranged between the pressure tube 2 and the stiffening layer 3. Thus, the stiffening layer 3 can be pressed radially outward by applying a pressure in the pressure tube 2 to stiffen the tubing apparatus 100. The pressure tube 2 has a radial width of 0.04 mm.

The inner tube 1 also comprises an outer polymer layer 34 which encases a monolithic reinforcement 35. The polymer layer 34 and reinforcement 35 of the inner tube 1 also have a radial width of 0.08 mm and 0.05 mm, respectively. A polytetrafluoroethylene coating 36 having a radial width of 0.03 mm is also provided on an inner surface of the inner tube 1 to optimize friction of surgical tools in a lumen of the inner tube 1. Thus, the overall wall thickness of the tubing apparatus 100 is 0.54 mm. The difference between an inner diameter 38 of the tubing apparatus 100 and an outer diameter 37 of the tubing apparatus 100 of 1.08 mm. These wall thicknesses are possible for all tubing apparatuses 100 with an inner diameter of 3 Fr to 18 Fr.

1. A tubing apparatus comprising a stiffening section comprising an inner tube, a stiffening layer and an outer tube, the stiffening layer being arranged concentrically outside the inner tube and the outer tube being arranged concentrically outside the stiffening layer the inner tube and the outer tube being radially rigid and the stiffening layer being radially movable, wherein a pressure can be built up between the inner tube and the stiffening layer in such a way that the stiffening layer can be pressed from the inside against the outer tube and thus stiffens the stiffening section under pressure.

2. The tubing apparatus according to claim 1, wherein an expandable pressure tube is arranged between the inner tube and the stiffening layer.

3. The tubing apparatus according to claim 1, wherein the stiffening layer comprises at least two stabilizing elements, the orientation of the stabilizing elements having at least a partial vector in the longitudinal direction of the tubing apparatus and the stabilizing elements.

4. The tubing apparatus according to claim 3, wherein the stabilizing elements are made of at least one of a metal and a plastic.

5. The tubing apparatus according to claim 1, wherein the inner tube and the outer tube are designed to be pressure-tight and are made of at least one of a plastic and a metal.

6. The tubing apparatus according to claim 1, wherein the tubing apparatus comprises a fluid coupling through which a fluid can be introduced into a region between the inner tube and the stiffening layer.

7. The tubing apparatus according to claim 1, wherein the tubing apparatus at least partially comprises a material visible in X-rays.

8. The tubing apparatus according to claim 1, wherein a front portion of the tubing apparatus is conical.

9. The tubing apparatus according to claim 1, wherein a hydrophilic or hydrophobic coating is arranged on an outside of the outer tube.

10. The tubing apparatus according to claim 1, wherein the stiffening section forms a part or all of the tubing apparatus.

11. The tubing apparatus according to claim 1, wherein the inner tube has an inner diameter of 1 mm to 11.333 mm.

12. The tubing apparatus according to claim 1, wherein the inner tube of the tubing apparatus comprises at least two lumens such that a plurality of devices can be inserted in separate lumens through the tubing apparatus.

13. A set comprising a tubing apparatus according to claim 1 and at least one dilator.

14. A method for selectively stiffening call the tubing apparatus according to claim 1, wherein the tubing apparatus is stiffened by introducing a fluid into the space between the inner tube and the stiffening layer.

15. The method according to claim 14, wherein by reducing the pressure in the space between the inner tube and the stiffening layer, the tubing apparatus becomes movable again.

16. A soft dilator comprising two lumens, a first lumen for a guide wire and a second lumen for a shaped wire, wherein the second lumen for the shaped wire is closed in the tip of the dilator and the first lumen for the guide wire is open.

17. The tubing apparatus according to claim 1, wherein the tubing apparatus is a gate apparatus for insertion into a body passage.

18. The tubing apparatus according to claim 2, wherein the pressure tube is at least one of elastic and unfoldable.

19. The tubing apparatus according to claim 3, wherein the stabilizing elements are displaceable relative to one another.

20. The tubing apparatus according to claim 4, wherein a plurality of stabilizing elements are braided to form a braided structure.

21. The tubing apparatus according to claim 6, wherein the fluid coupling is configured such that fluid can be introduced into the pressure tube.

22. The set according to claim 13, wherein the set comprises at least one soft dilator and at least one of a shaped wire and a guide wire.

23. The soft dilator according to claim 16, wherein the soft dilator is hydrophilically coated.

24. The soft dilator according to claim 16, wherein the soft dilator is softer than the shaped wire.

25. The soft dilator according to claim 16, wherein the soft dilator is visible under X-rays.
